(19) United States
(12) Patent Application Publication
(10) Pub. No.: US 2011/0184296 A1
(43) Pub. Date: Jul. 28, 2011

(54) METHOD AND SYSTEM FOR QUANTITATIVE ASSESSMENT OF CARDIAC ELECTRICAL EVENTS

(75) Inventors: Branislav Vajdic, Monte Sereno, CA (US); Aleksandar Mastilovic, Novi Sad (RS)

(73) Assignee: NEWCARDIO, INC., Santa Clara, CA (US)

(21) Appl. No.: 12/695,128

(22) Filed: Jan. 27, 2010

(51) Int. Cl. A61B 5/0402

(52) U.S. Cl. 600/509

(57) ABSTRACT

Systems and methods for characterizing aspects of an electrocardiogram signal are presented, wherein data quality and stability analysis paradigms are utilized to determine the timing certain cardiac electronic events with precision. In one embodiment, confidence factor calculation may be utilized to filter out nonusable ECG signals to leave a usable beat dataset, and this usable beat dataset may be utilized with moving window stability analysis to determine data most suitable extracted from a larger set to represent such larger set. The system may comprise a processor or microcontroller embedded into another system such as an electrocardiogram hardware system, personal computer, electrophysiology system, or the like.

### FIELD OF THE INVENTION

The present invention relates to the field of medical electronics. In particular, it concerns electronic systems, devices, and methods for acquisition, processing, and presentation of diagnostic data for use with humans and animals, such as electrocardiogram data.

### BACKGROUND

Although the electrocardiogram (frequently referred to as “ECG” or “EKG”) is a universally accepted diagnostic method in cardiology, frequent mistakes are made in interpreting ECGs, because the most common approach for interpretation of ECGs is based on human memorization of waveforms, rather than using vector concepts and basic principles of electrocardiography (see Hurst, J. W., Clin. Cardiol. 2000 January; 23(1):4-13). Another problem with traditional ECG recordings is that the ECG may not provide adequate indications of electrical activity of certain regions of the heart, especially the posterior region. The timing of cardiac electrical events, and the time intervals between two or more such events, has diagnostic and clinical importance. However, medical diagnosis and drug development has been significantly limited by the lack of adequate ECG measurement tools. Furthermore, prior analysis of ECG recordings required a substantial amount of training and familiarity with reading of the recorded waveforms. There have been many attempts to extract additional information from the standard 12-lead ECG measurement when measuring the electric potential distribution on the surface of the patient's body for diagnostic purposes. These attempts have included new methods of measured signal interpretation, either with or without introducing new measurement points, in addition to the standard 12-lead ECG points.

One of the oldest approaches, vector ECG (or “VCG”) includes the improvement of a spatial aspect to the ECG (see Frank, E., An Accurate, Clinically Practical System For Spatial Vectorcardiography, Circulation 13: 737, May 1956). Like conventional ECG interpretation, VCG uses a dipole approximation of electrical heart activity. The dipole size and orientation are presented by a vector that continuously changes during the heartbeat cycle. Instead of presenting signal waveforms from the measurement points (waveforms), as it is the case with standard 12-lead ECGs, in VCG, the measurement points are positioned in such a way that three derived signals correspond to three orthogonal axes (X, Y, Z), and these signals are presented as projections of the vector hodograph onto three planes (frontal, sagittal, and horizontal). In this way, VCG represents a step towards spatial presentation of the signal, but the cardiologist's spatial imagination skills were still necessary to interpret the ECO signals, particularly the connection to the heart anatomy. Furthermore, a time-dependence aspect (i.e., the signal waveform) is lost with this procedure, and this aspect is very important for ECG interpretation. VCG introduces useful elements which cannot be found within the standard 12-lead ECG, however, the incomplete spatial presentation and loss of the time dependence are major reasons why VCG, unlike ECG, has never been widely adopted, despite the fact that (in comparison to ECG) VCG can more often correctly diagnose cardiac problems, such as myocardial infarction.

There have been numerous attempts to overcome the drawbacks of the VCG method described above. These methods exploit the same signals as VCG (X, Y, Z), but their signal presentation is different than the VCG projection of the vector hodograph onto three planes. “Polarcardiogram” uses Aitoff cartographic projections for the presentation of the three-dimensional vector hodographs (see Sada, T., et al., J. Electrocardiol. 1982; 15(3):259-64). “Spherocardiogram” adds information on the vector amplitude to the Aitoff projections, by drawing circles of variable radius (see Niederberger, M., et al., J. Electrocardiol. 1977; 10(4):341-6). “3D VCG” projects the hodograph onto one plane (see Morikawa, J., et al., Angiology, 1987; 38(6):449-56. “Four-dimensional ECG” is similar to “3D VCG,” but differs in that every heartbeat cycle is presented as a separate loop, where the time variable is superimposed on one of the spatial variables (see Morikawa, J., et al., Angiology, 1996; 47: 1101-6.). “Chronotopocardiogram” displays a series of heart-activity time maps projected onto a sphere (see Titomir, L. I., et al., Int J Biomed Comput 1987; 20(4):275-82). None of these modifications of VCG have been widely accepted in diagnostics, although they have some improvements over VCG.

Electrocardiographic mapping is based on measuring signals from a number of measurement points on the patient's body. Signals are presented as maps of equipotential lines on the patient's torso (see McMechan, S. R., et al., J. Electrocardiol. 1995; 28 Suppl:184-90). This method provides significant information on the spatial dependence of electrocardiographic signals. The drawback of this method, however, is a prolonged measurement procedure in comparison to ECG, and a loose connection between the body potential map and heart anatomy.

Inverse epicardiac mapping includes different methods, all of which use the same signals for input data as those used in ECG mapping; and they are all based on numerically solving the so-called inverse problem of electrocardiography (see A. van Oosterom, Biomedizinisch Technik., vol. 42-El, pp. 33-36, 1997). As a result, distributions of the electric potentials on the heart are obtained. These methods have not resulted in useful clinical devices.

Cardiac electrical activity can be detected at the body surface using an electrocardiograph, the most common manifestation of which is the standard 12-lead ECG. Typical ECG signals are shown in present FIG. 1. The P-wave (2) represents atrial depolarization and marks the beginning of what is referred to as the “P-R interval”. The QRS complex (4) represents depolarization of the ventricles, beginning with QRS onset after the PR segment (5) and ending at a point known as the “J point” (6). Ventricular repolarization begins during the QRS and extends through the end of the Twave (14), at a point which may be termed “Tend” (8). The S-T segment (10) extends from the J point (6) to onset or start of the Twave (12). The Twave (14) extends from the Twave onset (12) through Tend (8). U waves (not shown) are present on some ECGs. When present, they merge with the end of the Twave or immediately follow it.

Physiologically, the Twave is the ECG manifestation of repolarization gradients, that is, disparities in degree of repolarization at a particular time point between different regions of the heart. It is likely that the Twave originates primarily from transmural repolarization gradient (see Yan and Antzelevitch; Circulation 1998; 98:1928-1936; Antzelevitch, J. Cardiovasc Electrophysiol 2003; 14:1259-1272.) Apico-basal and anteriorposterior repolarization gradients may also contribute (see Cohen I S, Giles W R, and Noble D; Nature. 1976; 262:657-661).

Transmural repolarization gradients arise because the heart's outer layer (epicardium) repolarizes quickly, the mid-myocardium repolarizes slowly, and the inner layer (endocardium) repolarizes in intermediate fashion. Referring again to FIG. 1, during the S-T segment (10), all layers have partially repolarized to a more or less equal extent, and the ST segment (10) is approximately isoelectric. A Twave (14) begins at a position which may be termed “Ton” (12), when the epicardial layer moves toward resting potential ahead of the other two layers. At the peak of the Twave (Tpeak) (16), epicardial repolarization is complete and the transmural repolarization gradient is at its maximum. Subsequently, endocardial cells begin their movement towards resting potential, thereby narrowing the transmural gradient and initiating the downslope of the Twave.

Finally, the M cells repolarize, accounting for the latter part of the Twave downslope. The Twave is complete at Tend (8) when all layers are at resting potential and the transmural gradient is abolished.

The QT interval (9) may be estimated from an ECG by measuring time from the end of the PR segment (5) to Tend (8). Abnormalities in the QT interval often mark susceptibility to life-threatening arrhythmias. Such abnormalities may be associated with genetic abnormalities, various acquired cardiac abnormalities, electrolyte abnormalities, and certain prescription and nonprescription drugs. An increasing number of drugs have been shown to prolong the QT interval and have been implicated as causes of arrhythmia. As a result, drug regulatory agencies are conducting increasingly detailed review of drug-induced abnormalities in cardiac electrical activity. The accuracy and precision of individual measurements is highly important for clinical diagnosis of heart disease and for evaluation of drug safety. Drug regulatory bodies worldwide now require detailed information regarding drug effects on cardiac intervals measured from ECG data (see M. Malik, PACE 2004; 27:1659-1669; Guidance for Industry: E14 Clinical Evaluation of QT/QTc Interval Prolongation and Proarrhythmic Potential for Non-Antiarrhythmic Drugs, http://www.fda.gov/cder/guidance/6922fnl.pdf).

Improved measurement accuracy and precision would reduce the risk of clinical error and the amount of resources required during drug development to meet regulatory requirements. This is particularly true for QT interval measurement. Problems in manual QT interval determination result in part from lead selection. Measured QT intervals can vary significantly depending upon the ECG lead selected for measurement. Another common problem is finding Tend. This is usually defined as the point at which the measured voltage returns to the isoelectric baseline. However, Twaves are often low-amplitude, morphologically abnormal, fused with a following U-wave, or obscured by noise. The same may apply to J-points, P-waves, U-waves and other important cardiac events.

Another fairly fundamental and important issue lies with selecting what data analyze. For example, it is convention in many hospitals and clinical study venues to utilize any three consecutive beats (from one ECG lead) from three successive ECG readings taken within a two minute time period. The challenge with such paradigm is that such data may or may not accurately represent the entire population of ECG signal activity for that particular patient. With the advent of Holter type monitors, more data is available, but in many clinical study settings, all of the data collected is not analyzed; rather, only selective portions of the data are examined, in accordance with whatever clinical study data analysis protocol is in place.

Referring to FIG. 1B, for example, notwithstanding approximately 24 hours or so of data which may be acquired by a Holter monitor in a given acquisition window (18), conventionally only selective time-based screening windows (22) are analyzed, and typically shorter extraction windows (24), or desired time windows during which cardiac electronic interval duration measurements (“IDMs”), such as QT, PR, RR (the “interbeat” interval from one R point to the next consecutive R point), and QRS intervals, are to be taken. Usually three ten-second ECGs are extracted from each extraction window (24), and within each ECG from each extraction window, IDM measurements are made on three consecutive beats. Thus, from each extraction window (24), a total of 9 IDMs might be collected (9-QT, 9-PR, 9-RR, and 9-QRS measurements), and this data needs to be collected for each extraction window (24) in the study, per study design. There are several approaches for selecting the timing of various screening windows (22) and extraction windows (24). The current state of the art in clinical studies calls for a human to examine a section of the data available from the Holter monitor or other device around the timing of a given predetermined extraction window (24), select the visually “most pleasing” three ten-second strips, and cause those 10-second data intervals to be extracted. The IDM measurements are then performed on these three consecutive ECG signals, and an average for an IDM for the three ECG set is reported as the IDM (e.g., QT interval) value for such extraction window (24).

Accurate and reproducible procedures for cardiac interval measurement are urgently needed. In particular, it would be valuable to have techniques and systems that involve less human subjective judgement, and take better advantage of the voluminous data available from modern collection systems, such as Holter type ECG collection systems. The subject invention addresses this challenge with a relatively noise-tolerant solution for determining the timing of cardiac electrical events.

### SUMMARY

One embodiment of the invention is directed to a method for processing ECG data acquired during an acquisition window time period, comprising: selecting a scanning window time period less than or equal to the acquisition time period; selecting a stability analysis window time period less than or equal to the scanning window time period; selecting an extraction window time period less than the stability analysis window time period; calculating a confidence factor for each beat comprising the ECG acquired during the scanning window time period; filtering out nonusable beats based upon the calculated confidence factors and a predetermined confidence threshold to arrive at a set of remaining usable beat data; conducting moving window stability analysis on stability windows of the usable beat ECG data, the stability windows defined by the stability analysis window time period, to numerically rank the stability windows in terms of stability; and extracting representative ECG data from a plurality of the best ranking, nonoverlapping, stability windows based upon extraction window positions within the stability windows defined by the extraction window time period. The scanning window time period may be about 10 minutes. The stability analysis window time period may be about 30 seconds, 60 sec, 90 sec, 120 sec, or any other arbitrary time period that is greater than the extraction window and less than the scanning window. The extraction window time period may be about 10 seconds, 20 sec, 30 sec, or any other arbitrary time period that is less than or equal to the chosen stability analysis window time period. Calculating a confidence factor may comprise establishing a confidence score based upon one or more factors selected from the group consisting of: an ECG signal confirmation factor, a noise level factor, and a curve fitting quality of measurement factor. The confidence factor may be expressed conveniently as a number between 0 and 100, and a predetermined confidence factor threshold may be about 0, 10, 20, 30, 40, 50, 60, 70, 80 or any number on the scale of 0 to 100, according to the discretion of the user. Conducting moving window stability analysis on stability windows of the usable beat ECG data may comprise creating a plurality of temporally adjacent and overlapping stability window datasets, and conducting a formula-based numerical stability analysis of beats captured within each of the plurality of stability window datasets. Conducting a formula-based numerical stability analysis may comprise calculating a plurality of IDMs (for example, RR, PR, QRS, or QT intervals, and the like) for each beat of the usable beat ECG data residing in each of the plurality of stability window datasets. Conducting a formula-based numerical stability analysis may further comprise calculating a standard deviation of IDMs (for example, RR, PR, QRS, or QT intervals, and the like) for all usable beat ECG data residing in each of the plurality of stability window datasets. Conducting a formula-based numerical stability analysis may further comprise calculating an IDM distance rank (for example, distance rank for RR, PR, QRS, or QT intervals, and the like) based upon the difference between a calculated average IDM value for a given stability window dataset relative to a mean or median IDM value for all usable beat ECG data residing within the entire scanning window time period. Conducting a formula-based numerical stability analysis may further comprise calculating a standard deviation of RR time values for all usable beats in each stability window dataset. Conducting a formula-based numerical stability analysis may further comprise calculating the number of usable beats in each stability window dataset. Representative ECG data may be extracted from the three best ranking stability windows, which are preferably nonoverlapping. The method may further comprise conducting statistical analysis based upon the extracted ECG data to determine what factors are responsible for variance in the ECG data in a population of patients. Statistical analysis may be conducted to determine whether a medicinal treatment is statistically responsible for variance in the ECG data in a population of patients, some of whom have been exposed to such medicinal treatment.

Another embodiment is directed to a system for processing ECG data acquired during an acquisition window time period, comprising a memory device configured to store data pertinent to one or more ECG signal waves sampled from electrodes operably coupled to one or more cardiac tissue structures during an acquisition window time period; and a processor operably coupled to the memory device and configured to controllably access the data, the processor configured to allow an operator to select a scanning window time period less than or equal to the acquisition time period, a stability analysis window time period less than or equal to the scanning window time period, and an extraction window time period less than the stability analysis window time period; calculate a confidence factor for each beat comprising the ECG acquired during the scanning window time period; filter out nonusable beats based upon the calculated confidence factors and a predetermined confidence threshold to arrive at a set of remaining usable beat data; conduct moving window stability analysis on stability windows of the usable beat ECG data, the stability windows defined by the stability analysis window time period, to numerically rank the stability windows in terms of stability; and extract representative ECG data from a plurality of the best ranking, nonoverlapping, stability windows based upon extraction window positions within the stability windows defined by the extraction window time period. The processor and memory device may be operably coupled to an analog signal acquisition system. The analog signal acquisition system may be operably coupled to one or more cardiac electrodes. The processor and memory device may be enclosed within an implantable housing. The implantable housing may be operably coupled to an external computing system and configured to exchange data with the external computing system by wire, or wirelessly. The analog signal acquisition system may comprise an ambulatory Holter monitor.

### BRIEF DESCRIPTION OF THE DRAWINGS

FIG. 1A illustrates aspects of a conventional ECG signal.

FIG. 1B illustrates aspects of a conventional ECG sampling and analysis paradigm.

FIG. 2A illustrates one embodiment of an acquisition window and scanning window configuration.

FIG. 2B illustrates a close-up depiction of one embodiment of a scanning window, stability window, and extraction window configuration.

FIG. 2C illustrates a close-up depiction of one embodiment of a stability window and extraction window configuration.

FIG. 3 illustrates one embodiment for carrying out filtration, stability analysis, and extraction in accordance with the subject invention.

FIG. 4 depicts an ECG system which may be integrated with aspects of the present invention.

FIG. 5 depicts an ambulatory Holter monitor system which may be integrated with aspects of the present invention.

FIG. 6 depicts an electrophysiology mapping system which may be integrated with aspects of the present invention.

FIG. 7 depicts an echocardiography system which may be integrated with aspects of the present invention.

FIGS. 8A and 8B depict fluoroscopy-based systems which may be integrated with aspects of the present invention.

### DETAILED DESCRIPTION

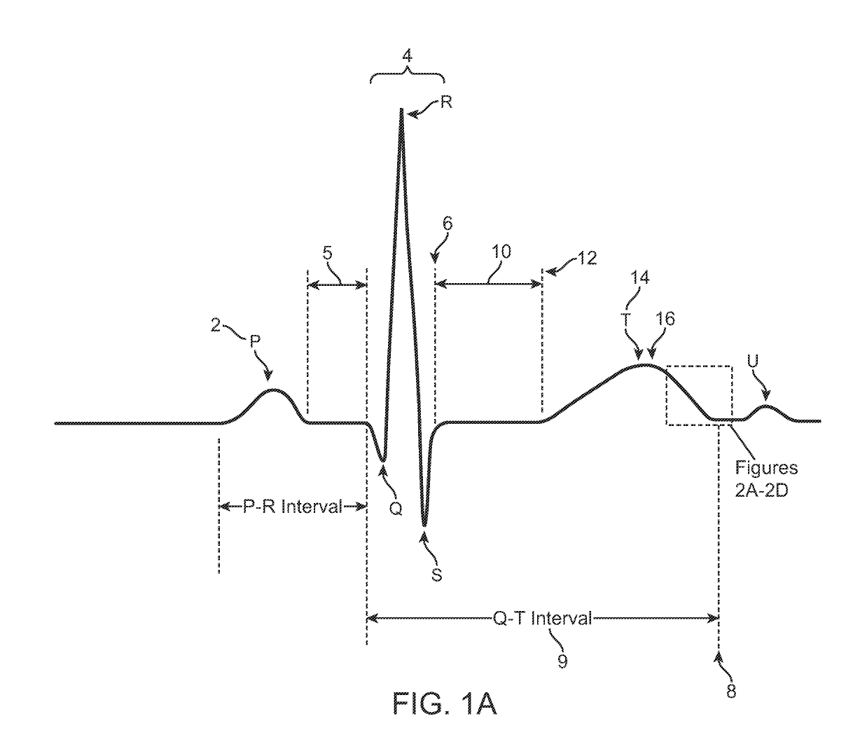

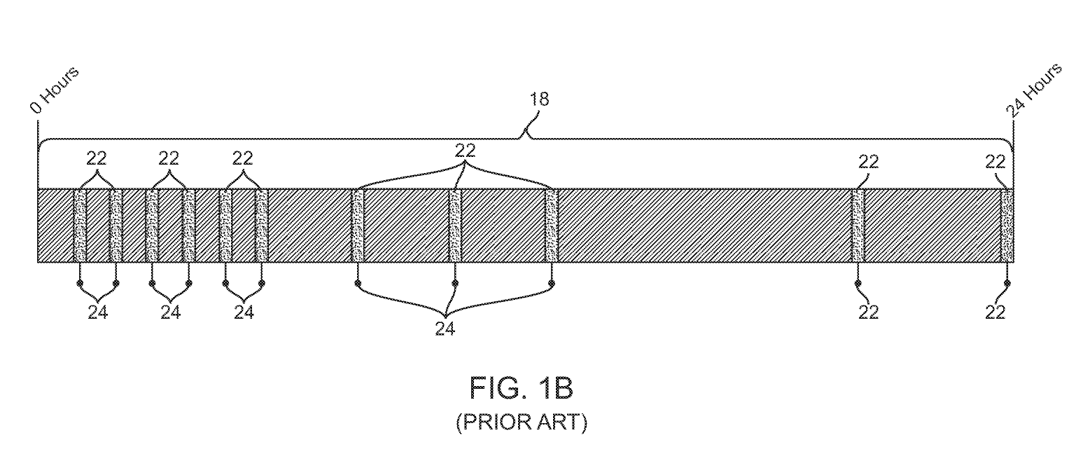

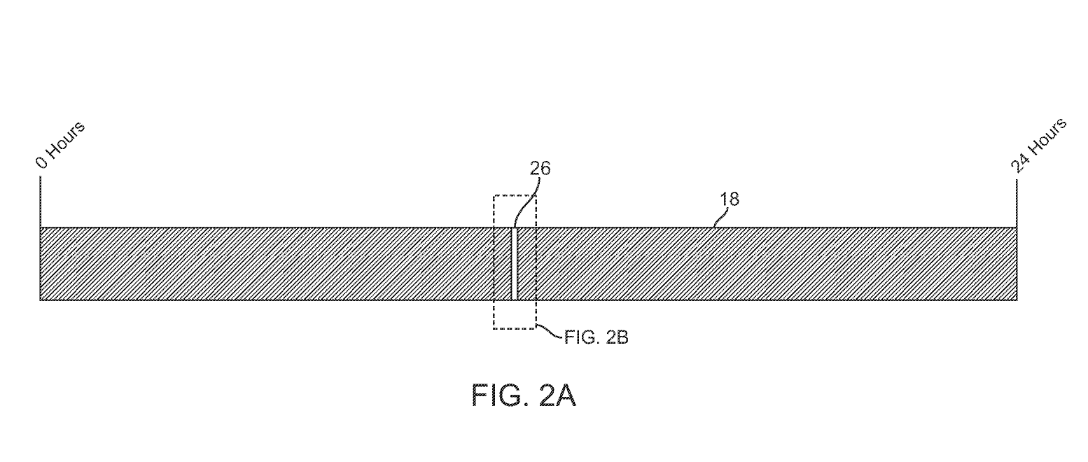

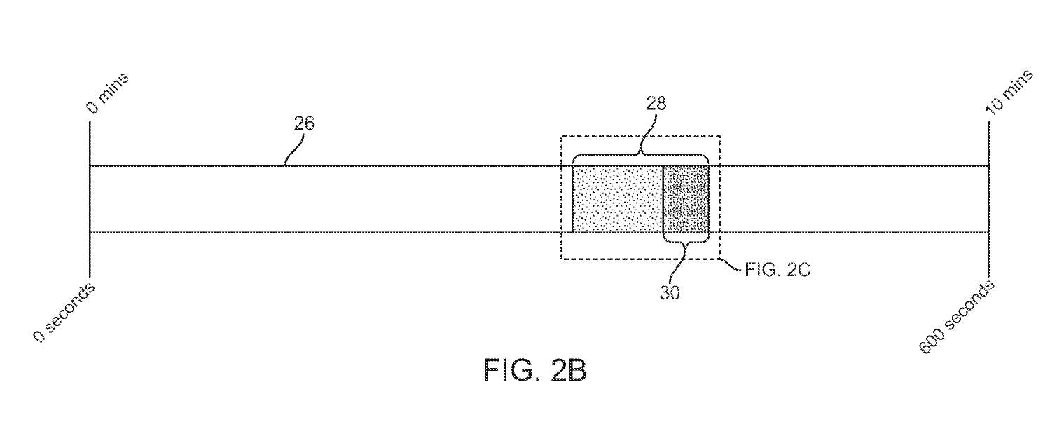

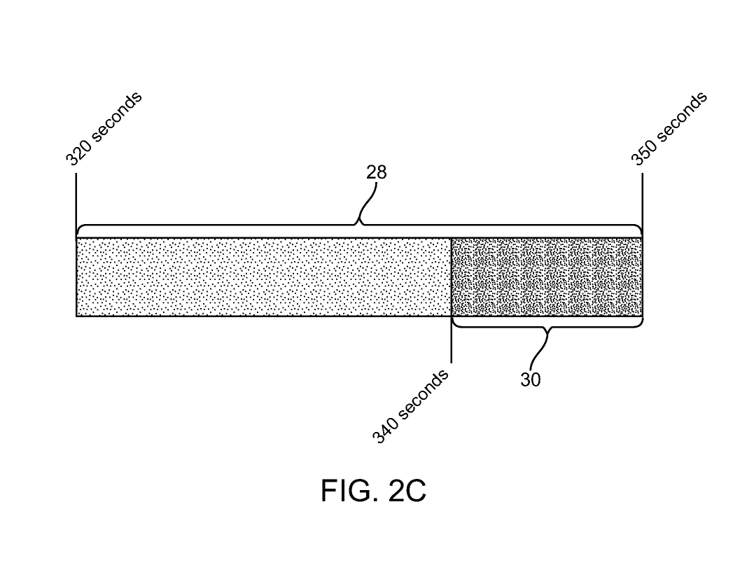

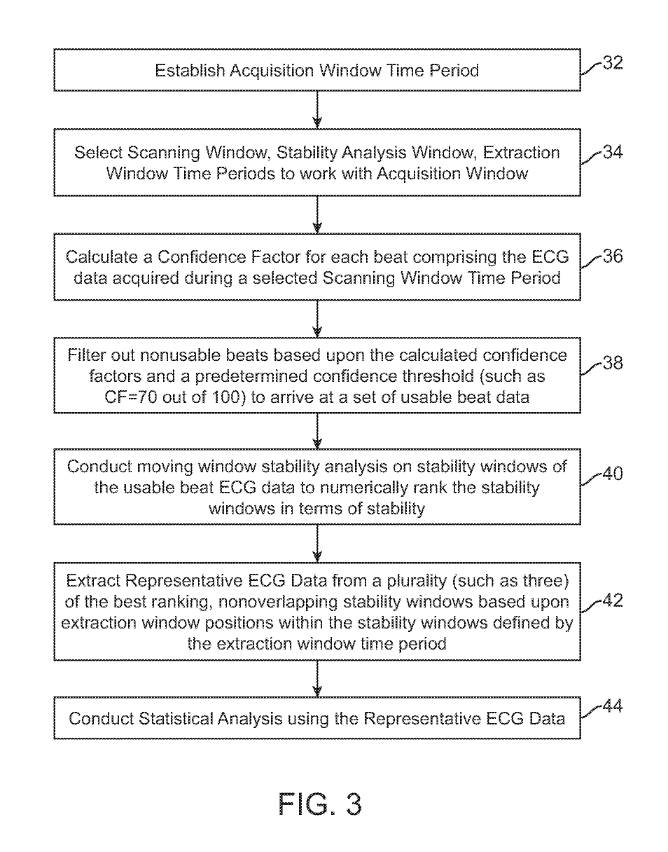

Referring to FIGS. 2A-3, techniques are described for automating certain aspects of ECG analysis to produce datapoints accurately representing the cardiac activity of a patient. FIGS. 2A-2C in particular are utilized to introduce terminology useful in describing the techniques illustrated in FIG. 3. Referring to FIG. 2A, a relatively long acquisition window (18), such as a 24 or 48 hour acquisition window, may be utilized to gather ECG data with a Holter type monitor or similar memory-enabled capture device. Within such acquisition window (18), a plurality of scanning windows (26) may be defined at times of clinical interest, similar to (or in some embodiments, identical to or a subset of) the screening windows (22) utilized conventionally and described in reference to FIG. 1B; alternatively, the entire run of data within the acquisition window (18) may be separated into a series of consecutive scanning windows (26). In one embodiment, each scanning window (26) may represent about 10 minutes of ECG data from the larger acquisition window (18). Referring to FIG. 2B, an individual scanning window (26) is depicted in a magnified view. The scanning window (26) may be divided into a plurality of overlapping stability windows (28), preferably equal in duration and which themselves contain shorter extraction windows (30). Referring to FIG. 2C, an even closer view illustrates that in one embodiment, for example, a stability window (28) period of about 30 seconds may be selected, the last about 10 seconds of which may be defined as the extraction window (30). The particular stability window (28) featured in FIG. 2C contains data collected between the 320^{th} second of the scanning window (26) of FIG. 2B and the 350^{th} second of the scanning window (26) of FIG. 2B, with the extraction window (30) representing data from the 340^{th} second of the scanning window of FIG. 2B to the 350^{th} second of the scanning window (26) of FIG. 2B. In different terminology, the stability window (28) of FIG. 2C may represent [320 s, 350 s] of the scanning window (26) of FIG. 2B, while the extraction window represents [340 s, 350 s] of the scanning window (26) of FIG. 2B. A series of overlapping and adjacent stability windows may be defined within a broader scanning window using a paradigm as follows: a first stability window at [0 s, 30 s]; a second stability window at [1 s, 31 s]; a third stability window at [2 s, 32 s], and so on. Even smaller shifts in the stability window are possible and may be desirable, for example a first stability window at [0 s, 30 s]; a second stability window at [0.1 s, 30.1 s]; a third stability window at [0.2 s, 30.2 s], and so on. Larger shifts also may be utilized, for example, a first stability window at [0 s, 30 s]; a second stability window at [2 s, 32 s] and so on. Larger or smaller shifts in the stability window may be chosen essentially at the discretion of the user, for example, it may be 2 s, 4 s, 10 s, 20 s, 30 s, or even more. Such a paradigm of breaking a larger acquisition window (18) into scanning windows (26), stability windows (28), and extraction windows (30) may be utilized to accurately and repeatably process ECG data, as described in reference to FIG. 3.

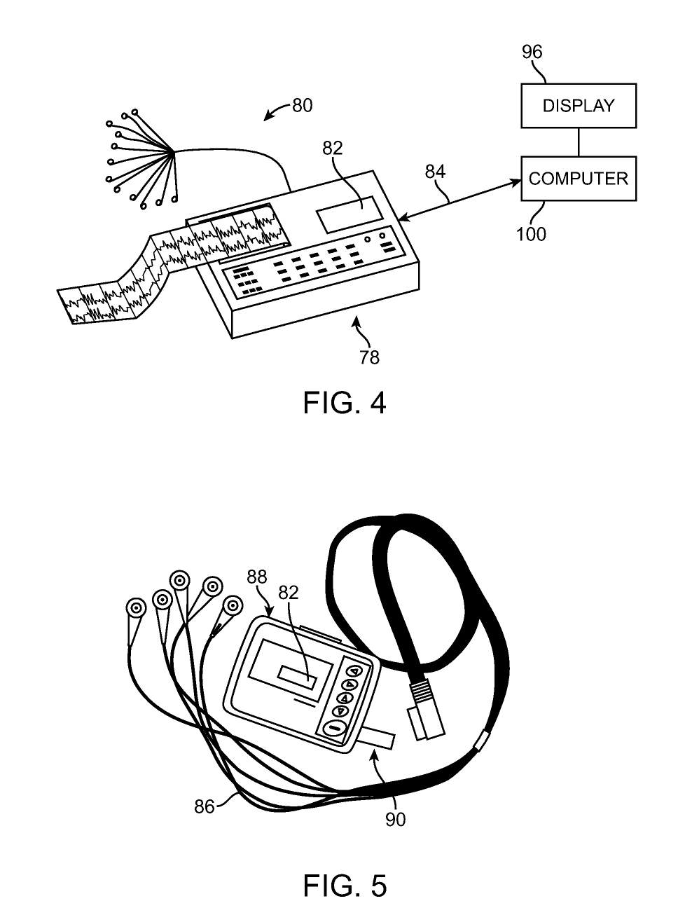

Referring to FIG. 3, after establishing an acquisition window time period (32), such as 24 or 48 hours for ECG acquisition using a configuration such as a Holter system or other variations as described below in reference to FIGS. 4-8B, scanning window, stability analysis window, and extraction window time periods may be selected (34), such as 10 minutes for each scanning window, positioned as per a related clinical study protocol or the like, 30 seconds for each overlapping stability analysis window, staged at consecutive seconds in time as in the aforementioned example, and ten seconds for each extraction window residing at the end of each stability analysis window. For all of the ECG data in each scanning window, confidence factor analysis (36) may be conducted to separate or filter out nonusable ECG signal cycles, or “beats”, from usable beats (38) with pass muster under such confidence factor analysis. Suitable confidence factor analysis techniques are described, for example, in U.S. patent application Ser. No. 12/184,068, which is incorporated by reference herein in its entirety. In one embodiment, such confidence factor analysis may involve three parts: a) analyzing each given beat signal to determine if such signal pattern even represents an ECG signal; if not, such beat is given a low confidence factor score; b) examining noise components of the overall given beat signal pattern; for example, high frequency noise may represent radiofrequency interference, while lower frequency “signal wandering”, such as in a sinusoidal pattern, may also represent noise; a quantitative score is assigned pertinent to the levels of noise in the signal for such beat, with lower scores representing more noisy beats; c) a quality of measurement score is assigned, higher representing higher beat quality, based upon curve fitting analysis; for example, in one embodiment, least squares curve fitting may be utilized to fit a curve to a beat signal, variance checked for fit quality, and iterations conducted to improved fit quality until a threshold level of goodness of fit is achieved; a score is assigned for such goodness of fit. As described in the aforementioned incorporated by reference application, utilizing such analysis paradigm, most drug study ECG beat signals pass with a confidence factor score of greater than 70 out of a maximum 100 (if a 0 to 100 scale for confidence factor score is used; the CF scale is arbitrarily chosen because 0-100 is convenient); the remaining 1-4% of beats are rejected. Thus, referring again to FIG. 3, in one embodiment beats with confidence factor scores less than CF=70 may be rejected to arrive at a set of usable beat data (38).

Subsequent to filtering to create the subset of usable beat data, moving window stability analysis may be conducted on the usable beat dataset to numerically rank each of the stability windows in terms of data stability (40). In one embodiment, a simple formula may be utilized to create an overall stability score for each stability window:

Stability Rank=M*(StdDev(QTc) rank)+N*(QTc distance rank)+O*(StdDev(RR) rank)+P*(number of beats rank)

In the above formula, “StdDev(QTc)” represents the standard deviation of corrected QT interval values of all usable beats in a given stability window; the window having the lowest StdDev(QTc) will get the lowest (best) rank. “Corrected QT interval values”, “Corrected QT”, and “QTc” are all used interchangeably to mean the measured QT interval value corrected for heart rate, for example by Bazett's formula (QTc=QT/(RR)^{0.5}, Fridercia's formula (QTc=QT/(RR)^{0.33}, or individualized rate correction, the application of which are well known to those of ordinary skill in the art. In the above formula, “QTc distance” is a rank based on the difference between an average (mean or median) QTc value for the stability window and the mean or median QTc value of all usable beats within a given scanning window. The stability window having the shortest distance (smallest difference) from such mean or median value shall be assigned the lowest (best) rank. In the above formula, “StdDev(RR)” represents the standard deviation of RR interval values of all usable beats in a given stability window. The window having the lowest StdDev(RR) is assigned the lowest (best) rank. In the above formula, the “number of beats rank” simply represents the number of usable beats in a given stability window. The stability window with the highest number of such usable beats would be assigned the lowest (best) rank. In one embodiment, the weighting coefficient variables M, N, 0, and P may be assigned the values 3, 3, 1, and 1, respectively. Any other value including 0 may be assigned to any of the weighting coefficients at the discretion of the user. Once the above formula is applied and ranks are calculated, in one embodiment, the system is configured to select the three non-overlapping (meaning that the extraction windows to not overlap) windows having the highest such formula based ranks (i.e., the lowest sum of the ranks)—and use such data for extraction (42), and subsequent statistical analysis (44).

In an alternative embodiment, all usable beats in a given scanning window (such as all 600 seconds of the scanning window 26 of FIG. 2B) may be processed, and all QTc values for all usable beats may be calculated. Mean and median values for this array of QTc values may be calculated, and a simple decision making function may be used to select the three (or whatever selected subset number) best nonoverlapping 10 second (or whatever selected extraction window time period) strips for extraction and subsequent statistical analysis. In such embodiment, every such 10 second strip is being evaluated based upon its average QTc value, and will be compared to the mean and median of the distribution of all usable beats in the scanning window. Thus in such embodiment, the following formula may be utilized:

Selection Rank=absolute value of (QTmean[extraction candidate]−QTmean[scanning window])+absolute value of (QTmean[extraction candidate]−QTmean[scanning window])

As in the aforementioned stability formula, weighting coefficients may be assigned to both terms of the above Selection Rank formula. Using this formula, all 10 second (or whatever time period is selected) extraction candidates may be ranked, and the top three (or whatever selected representative number) non-overlapping rank values (i.e., with the lowest calculated selection rank values) may be chosen to be the representative ECG data for subsequent statistical analysis, as in the last two steps of the embodiment described in reference to FIG. 3 (42, 44).

In practice, the techniques described in reference to FIGS. 2A-3 may be conducted on one or more computing systems, such as a personal computer, utilizing customized software, semi-customized software based, for example, on spreadsheets or customized configurations in applications such as the software package available under the tradename LabView® by National Instruments, Inc., and/or hardware configured to run embedded software. In some embodiments, it is preferred to have pertinent systems electronically integrated to facilitate realtime or near-realtime analysis in accordance with the techniques described above. For example, referring to FIG. 4, in one embodiment, an ECG acquisition system (78) and associated electrodes (80) preferably are integrated with a computer (100) using a wired or wireless coupling (84) whereby the computer (100) may receive and/or request data from the ECG system (78), and control activities and/or receive information from an embedded device (88), such as a card comprising integrated circuits and/or memory (and in one embodiment housed in a card housing and comprising an electromechanical card interface to connect with a bus comprising the ECG system), an application specific integrated circuit (“ASIC”), or a field programmable gate array (“FPGA”), each of which preferably would be configured to conduct confidence factor based filtration, moving window stability analysis, and/or representative ECG data extraction using raw data received by the ECG system (78) from the electrodes (80), in accordance with any instructions or control sequences that may be received from the computer (100), should the computer be connected at the time of sampling or before sampling. Referring to FIG. 5, an ambulatory, portable, Holter style ECG system (88) may also be similarly coupled to an embedded device (82) configured to conduct such filtration, analysis, and/or extraction using raw data received by such system (88) from an operably coupled electrode set (86). A bus or connector (90) may be provided for computing system (not shown) connectivity.

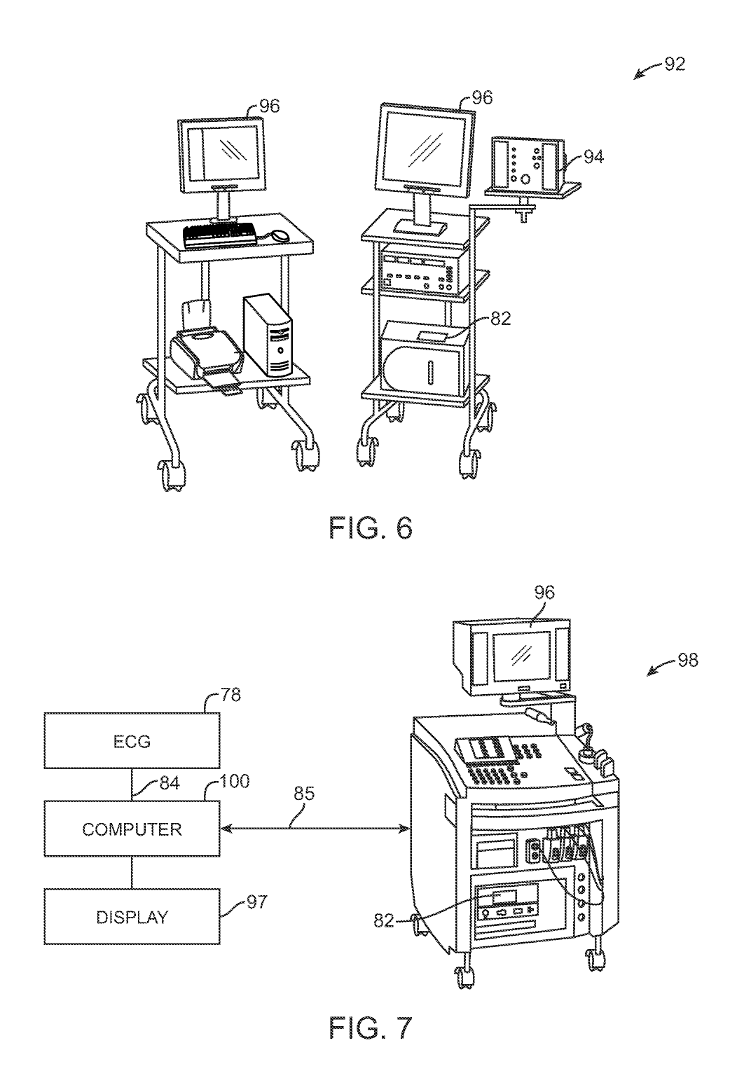

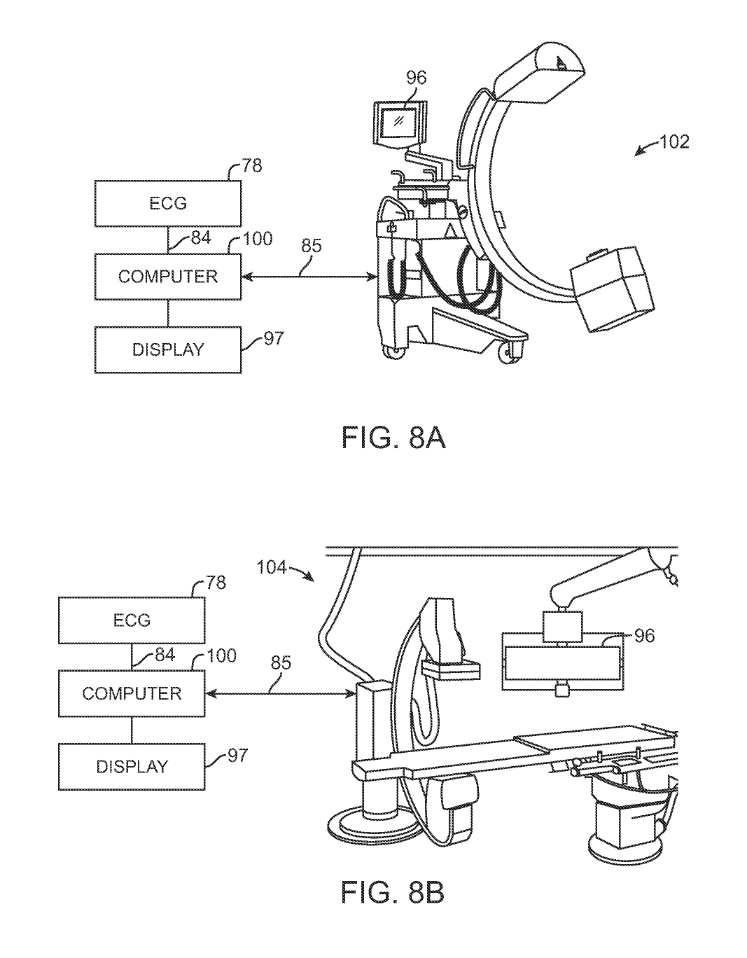

Referring to FIGS. 6-8B, other medical information processing systems commonly associated with ECG signal processing may also be desirably integrated with or embedded with processing infrastructure configured to conduct filtration, analysis, and/or extraction, in accordance with the present invention. For example, referring to FIG. 6, an electrophysiology mapping system (92), such as those available from Biosense Webster under the tradename CartoXP®, may also be operably coupled to an embedded device (82) configured to conduct filtration, analysis, and/or extraction using raw data received by such system (92) from an operably coupled electrode set (not shown) coupled to an electrode connectivity bus panel (94). Results from such processing may be directed to the one or more displays (96). Referring to FIG. 7, an echocardiography system (98), such as those available from Siemens Medical Systems, Inc. under the tradename Sequoia®, may be operably coupled to a computing system (100) and an ECG system (78). An embedded device (82) configured to conduct filtration, analysis, and/or extraction using raw data received from the ECG system (78), may be coupled to any one of the ECG system (78), as in FIG. 4, the computing system (100), or the echocardiography system (98). Data pertinent to the filtration, analysis, and/or extraction preferably may be directed to either of the echocardiography display (96) or the computing system display (97). Similarly, referring to FIGS. 8A and 8B, a relatively simple fluoroscopy system (102), such as that depicted in FIG. 8A, or a more complex angiography system (104), such as that depicted in FIG. 8B, may be operably coupled and/or embedded with a device configured to conduct filtration, analysis, and/or extraction using raw data received by electrodes operably coupled to a computing system (100), associated ECG system (78), the embedded device, or other system. Connectivity of the various components of such system configurations, such as the processor, memory device, and operating room electronic device, may be conducted using Ethernet and/or communication protocols such as TCPIP, FTP, or HTTP.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A method for processing ECG data acquired during an acquisition window time period, comprising:
a. selecting a scanning window time period less than or equal to the acquisition time period;
b. selecting a stability analysis window time period less than or equal to the scanning window time period;
c. selecting an extraction window time period less than the stability analysis window time period;
d. calculating a confidence factor for each beat comprising the ECG acquired during the scanning window time period;
e. filtering out nonusable beats based upon the calculated confidence factors and a predetermined confidence threshold to arrive at a set of remaining usable beat data;
f. conducting moving window stability analysis on stability windows of the usable beat ECG data, the stability windows defined by the stability analysis window time period, to numerically rank the stability windows in terms of stability; and
g. extracting representative ECG data from a plurality of the best ranking, nonoverlapping, stability windows based upon extraction window positions within the stability windows defined by the extraction window time period.

2. The method of claim 1, wherein the scanning window time period is about 10 minutes.

3. The method of claim 1, wherein the stability analysis window time period is about 30 seconds.

4. The method of claim 3, wherein the extraction window time period is about 10 seconds.

5. The method of claim 1, wherein calculating a confidence factor comprises establishing a confidence score based upon one or more factors selected from the group consisting of: an ECG signal confirmation factor, a noise level factor, and a curve fitting quality of measurement factor.

6. The method of claim 1, wherein the predetermined confidence threshold is about 70 on a scale of 0 to 100.

7. The method of claim 1, wherein conducting moving window stability analysis on stability windows of the usable beat ECG data comprises creating a plurality of temporally adjacent and overlapping stability window datasets, and conducting a formula-based numerical stability analysis of beats captured within each of the plurality of stability window datasets.

8. The method of claim 7, wherein conducting a formula-based numerical stability analysis comprises calculating a corrected QT time for each beat of the usable beat ECG data residing in each of the plurality of stability window datasets.

9. The method of claim 8, wherein conducting a formula-based numerical stability analysis further comprises calculating a standard deviation of corrected QT time values for all usable beat ECG data residing in each of the plurality of stability window datasets.

10. The method of claim 9, wherein conducting a formula-based numerical stability analysis further comprises calculating a corrected QT distance rank based upon the difference between a calculated average corrected QT value for a given stability window dataset relative to a medial corrected QT value for all usable beat ECG data residing within the entire scanning window time period.

11. The method of claim 10, wherein conducting a formula-based numerical stability analysis further comprises calculating a standard deviation of RR time values for all usable beats in each stability window dataset.

12. The method of claim 11, wherein conducting a formula-based numerical stability analysis further comprises calculating the number of usable beats in each stability window dataset.

13. The method of claim 1, wherein representative ECG data is extracted from the three best ranking, nonoverlapping stability windows.

14. The method of claim 13, further comprising conducting statistical analysis based upon the extracted ECG data to determine what factors are responsible for variance in the ECG data in a population of patients.

15. The method of claim 14, wherein statistical analysis is conducted to determine whether a medicinal treatment is statistically responsible for variance in the ECG data in a population of patients, some of whom have been exposed to such medicinal treatment.

16. A system for processing ECG data acquired during an acquisition window time period, comprising:
a. a memory device configured to store data pertinent to one or more ECG signal waves sampled from electrodes operably coupled to one or more cardiac tissue structures during an acquisition window time period; and
b. a processor operably coupled to the memory device and configured to controllably access the data, the processor configured to:
1) allow an operator to select a scanning window time period less than or equal to the acquisition time period, a stability analysis window time period less than or equal to the scanning window time period, and an extraction window time period less than the stability analysis window time period;
2) calculate a confidence factor for each beat comprising the ECG acquired during the scanning window time period;
3) filter out nonusable beats based upon the calculated confidence factors and a predetermined confidence threshold to arrive at a set of remaining usable beat data;
4) conduct moving window stability analysis on stability windows of the usable beat ECG data, the stability windows defined by the stability analysis window time period, to numerically rank the stability windows in terms of stability; and
5) extract representative ECG data from a plurality of the best ranking, nonoverlapping, stability windows based upon extraction window positions within the stability windows defined by the extraction window time period.

17. The system of claim 16, wherein the processor and memory device are operably coupled to an analog signal acquisition system.

18. The system of claim 17, wherein the analog signal acquisition system is operably coupled to one or more cardiac electrodes.

19. The system of claim 16, wherein the processor and memory device are enclosed within an implantable housing.

20. The system of claim 19, wherein the implantable housing is operably coupled to an external computing system and configured to exchange data with the external computing system by wire, or wirelessly.

21. The system of claim 17, wherein the analog signal acquisition system comprises an ambulatory Holter monitor.
